(19) United States
(12) Patent Application Publication
(10) Pub. No.: US 2011/0060008 A1
(43) Pub. Date: Mar. 10, 2011

(54) PHARMACEUTICAL COMPOSITION CONTAINING ACETYLCHOLINE ESTERASE INHIBITOR AND METHOD FOR THE PREPARATION THEREOF

(76) Inventors: Deepak Murpani, Pallini (GR); Panagiotis Paterakis, Pallinii (GR)

(21) Appl. No.: 12/666,508

(22) PCT Filed: Jun. 26, 2008
(86) PCT No.: PCT/GR08/00049

§ 371 Date: Aug. 19, 2010

(30) Foreign Application Priority Data
Jun. 26, 2007 (GR) 20070100405

(51) Int. Cl. A61K 31/4353; A61P 25/28

(52) U.S. Cl. 514/319

(57) ABSTRACT

A pharmaceutical composition for oral administration comprising donepezil or pharmaceutically acceptable salt thereof, and an effective amount of pH dependent excipient as a taste masking agent, to suppress the release of donepezil in the pH environment of the oral cavity and increase the release of donepezil in acidic environment.

### TECHNICAL FIELD OF THE INVENTION

The present invention relates to improved pharmaceutical compositions for oral administration and in particular to an orally disintegrating composition comprising a therapeutically effective quantity of an acetylcholine esterase inhibitor, Donepezil or pharmaceutically acceptable salts thereof, and a method for the preparation thereof.

### BACKGROUND OF THE INVENTION

Donepezil hydrochloride is a well-known medicinal active ingredient with a strong and highly selective acetylcholine esterase inhibiting activity, and has been found as an efficient drug for the treatment of dementia and Alzheimer's disease. Its cholinergic enhancement property is considered to be the reason for the alleviation of symptoms in patients. Donepezil hydrochloride is a reversible inhibitor of the enzyme acetylcholinesterase, known chemically as (±)-2,3-dihydro-5,6-dimethoxy-2-[[1-(phenylmethyl)-4-piperidinyl]methyl]-1H-inden-1-one hydrochloride. It has an empirical formula of C24H29NO3HCl. Donepezil hydrochloride is a white crystalline powder and is freely soluble in chloroform, soluble in water and in glacial acetic acid, slightly soluble in ethanol and in acetonitrile and practically insoluble in ethyl acetate and in n-hexane. Donepezil hydrochloride is currently available under the trade name ARICEPT® for oral administration in film-coated and orally disintegrating tablets containing 5 or 10 mg of donepezil hydrochloride. Donepezil hydrochloride has an unpleasant bitter taste and numbness, and the oral administration of said drug puts a burden on a patient and lowers compliance.

Various methods are already known for the industrial preparation of oral dosage forms comprising Donepezil or pharmaceutically acceptable salts thereof, as an active ingredient due to its useful therapeutic properties. However, the prior art has encountered substantial difficulties in the production of the oral solid formulations of a desirable taste due to complicated manufacturing processes and quality problems. Further, the introduction of flavours or sweeteners into the formulation gives little improvement, especially when incorporated with highly soluble drugs, such as donepezil, and the layer coating on the surface of the granules of the drug for taste masking purposes requires special and expensive equipment and very complicated manufacture processes.

EP-B-974 366 discloses a pharmaceutical composition which comprises an active ingredient with unpleasant taste and an anionic acidic polysaccharide, such as carrageenan chondroitin sulfate, dextran sulfate, alginic acid, gerun gum, xanthan gum and their salts in order to reduce the bitter taste. In addition, in long-lasting treatments such as dementia treatment, Alzheimer's disease, it is very important that the compositions are easy administrated to swallow, as said medicaments are for people having insufficient swallowing functions, e.g. elderly people. For this purpose, orally disintegrating dosage forms are used, which disintegrates within 90 seconds.

EP-A-1 025 858 discloses a pharmaceutical composition comprising an active ingredient with unpleasant taste and a taste masking substance such as povidone and/or copolyvidone. Moreover, the manufacture process using granulation with polymeric substances such as PVP, copovidone has also been proved difficult to perform and the taste improvement is not sufficient.

EP-A-1 260 215 discloses quickly disintegrating tablets which are prepared by blending an active ingredient with a saccharide and polyvinyl alcohol. The active ingredient is mixed with high amounts of saccharide (about 80% wt/wt), and the resulting mixture is kneaded with water including polyvinyl alcohol dissolved therein or in an organic solvent, and subjected to compression-molding. The process, however, usually involves employing huge amounts of saccharides and alcohols as solvents which may result in conversion of polymorphs or formation of impurities.

Although the above patents represents an attempt to overcome the problems associated with pharmaceutical compositions comprising donepezil or its pharmaceutically acceptable salts, there still exists a need for improving the unpleasant taste of such pharmaceutical compositions.

### OBJECT OF THE INVENTION

The object of the present invention is to provide a pharmaceutical composition of donepezil or pharmaceutically acceptable salts thereof, with improved taste and also suppress release of donepezil in the oral cavity and improve release in acidic environment. The taste of donepezil or pharmaceutically acceptable salt is improved with the use of pH dependent excipient.

Yet another object of the present invention is to provide a solid oral dosage form and the process for the preparation of donepezil or pharmaceutically acceptable salt and an effective amount of pH dependent excipient as a taste masking agent to suppress release of donepezil in the oral cavity and improve release in acidic environment.

### SUMMARY OF THE INVENTION

The present invention provides a pharmaceutical composition for oral administration comprising donepezil or pharmaceutically acceptable salt thereof, and an effective amount of pH dependent excipient as a taste masking agent, to suppress the release of donepezil in the pH environment of the oral cavity and increase the release of donepezil in acidic environment.

The composition of the present invention overcomes the deficiencies of the existing products and increases patient compliance.

A further aspect of the present invention is to provide a method for the preparation of a solid dosage formulation for oral administration containing donepezil or pharmaceutically acceptable salts thereof as which disintegrates fast without leaving an unpleasant taste in the mouth, thereby improving the pharmacotechnical characteristics of the composition which may be prepared in a simple and cost efficient manner.

The present invention also provides a process for preparing solid oral dosage form of donepezil or pharmaceutically acceptable salt thereof with an effective amount of pH dependent excipient as a taste masking agent, to suppress the release of donepezil in the pH environment of the oral cavity and increase the release of donepezil in acidic environment, said process comprising

(a) dissolving/dispersing donepezil or pharmaceutically acceptable salt in water/acidic medium;
(b) adding to the solution/suspension formed in step (a) an effective amount of said pH dependent excipient; and
(c) formulating with excipient(s) into a solid oral dosage form.
Steps (a) and (b) may be optionally reversed

The product of the present invention may be an orally disintegrating tablet comprising donepezil or pharmaceutically acceptable salt thereof and pH dependent excipient, polacrilin potassium.

More specifically, the present invention provides a pharmaceutical composition for oral administration comprising donepezil or pharmaceutically acceptable salt thereof, and an effective amount of polacrilin potassium as a taste masking agent, wherein the weight ratio of donepezil or pharmaceutically acceptable salt thereof to polacrilin potassium is 1:1 to 1:6, to suppress the release of donepezil in the pH environment of the oral cavity and increase the release of donepezil in acidic environment.

The process for the preparation of a solid oral dosage form of donepezil or pharmaceutically acceptable salt thereof and an effective amount of polacrilin potassium as a taste masking agent, wherein the weight ratio of donepezil or pharmaceutically acceptable salt thereof to polacrilin potassium is 1:2 to 1:6, to suppress the release of donepezil in the pH environment of the oral cavity and increase the release of donepezil in acidic environment, said process comprising

(a) dissolving/dispersing donepezil or pharmaceutically acceptable salt in water/acidic medium; (b) adding to the solution/suspension formed in (a) an effective amount of polacrillin potassium; and (c) formulating with excipient(s) into a solid dosage form. Steps (a) and (b) may be optionally reversed

### DETAILED DESCRIPTION OF THE INVENTION

Donepezil has taste characteristics which when administered orally without any excipients, render donepezil unpalatable to a subject. The present invention provides a pharmaceutical composition for oral administration of donepezil or pharmaceutically acceptable salt with an improved taste.

According to one embodiment of the present invention a pharmaceutical composition of donepezil or pharmaceutically acceptable salt thereof with improved taste is prepared with a pH dependent excipient. The pharmaceutical composition for oral administration of the present invention comprises donepezil or pharmaceutically acceptable salt thereof, and an effective amount of pH dependent excipient as a taste masking agent, to suppress the release of donepezil in the pH environment of the oral cavity and increase the release of donepezil in acidic environment. The effective amount of pH dependent excipient may be defined by the weight ratio of donepezil to pH dependent excipient from 1:15 to 2:1, preferably in the range 1:1 to 1:6

It has been surprisingly found that the object of the present invention is achieved by employing pH dependent excipients which may be selected from ion exchange resin such as Polacrilin Potassium or methacrylic acid copolymer such as Eudragit E as a taste masking agent.

Ion exchange resins are acidic or basic functional groups and have the ability to exchange counter ions with aqueous solution surrounding them. Specifically, Polacrilin Potassium is a weakly acidic cation exchange resin, and has the ability to bind considerable quantities of water due to its hydrophilic nature. Polacrilin potassium forms a complex (also referred to as resinate) with donepezil or pharmaceutically acceptable salt thereof. Due to the complexation between Polacrilin potassium and donepezil, donepezil is not released in the mouth, so the patient does not feel the unpleasant taste of the drug when it is swallowed. When donepezil resinate comes into contact with the gastrointestinal fluids, such as the acid of the stomach, donepezil is released from resinate directly into solution and then absorbed. The resin passes through the GI tract without being absorbed. Thus, Polacrilin Potassium serves as a protective barrier, suppressing release of the active ingredient in the pH environment of the oral cavity and increase release in acidic environment. The effective amount of polacrilin potassium may be defined by the weight ratio of donepezil to polacrilin potassium from 1:15 to 2:1, preferably in the range 1:1 to 1:6

On formation of the donepezil polacrilin potassium resinate, additional excipients may be added such as fillers, binders, compression aids, disintegrants, glidants, lubricants, flavouring agents, water scavengers, colorants, sweetening agents, coating agents and preservatives. Although the pharmaceutical composition may be in various forms, the preferred solid forms are tablets, oral dispersible tablets, soluble tablets, water dispersible tablets, sprinkles, chewable tablets, effervescent tablets, orally disintegrating tablets, powder for suspension, capsules, sachets and caplets.

The preferred pharmaceutical compositions of donepezil or pharmaceutically acceptable salt thereof are in the form of solid dosage forms such as orally disintegrating tablets and the like, in all shapes and sizes, coated or uncoated. The pharmaceutical composition of the present invention when formulated into orally disintegrating tablets disintegrates within 90 seconds as measured by the in vitro disintegration test according to Ph.Eur. The composition according to the invention preferably disintegrates in less than 60 s, and more preferably in less than 30 s. The pharmaceutical composition of the present invention comprising donepezil or salts thereof, disintegrates fast without leaving an unpleasant taste in the mouth and which show a good physicochemical stability and low friability rendering them suitable for normal packaging and storing procedures.

The present invention provides a solid dosage formulation for oral administration containing donepezil or pharmaceutically acceptable salts thereof is effective with sufficient shelf-life, good pharmacotechnical properties and bioavailability of donepezil. The improved solid pharmaceutical composition of the present invention is characterized by physicochemical properties suitable for the tablet formulation by wet granulation, so as to obtain the adequate release rate of donepezil. Another essential advantage of the present invention is that the solid dosage form according to the present invention ensures excellent stability and bioavailability of the active ingredient. The manufacturing process for preparation according to the present invention is simpler and inexpensive in comparison to any other conventional method.

Another pH dependent excipient which may be used in the present invention is Eudragit E which is a cationic polymer based on dimethylaminoethyl methacrylate and neutral methacrylates. It is soluble in gastric fluid below pH 5 and in weakly acidic buffer solutions. The effective amount of Eudragit E may be defined by the weight ratio of donepezil to Eudragit E from 1:15 to 2:1, preferably in the range 1:1 to 1:6.

According to yet another embodiment of the present invention is provided a process for the preparation of a solid oral dosage form of donepezil or pharmaceutically acceptable salt thereof, said process comprising

(a) dissolving/dispersing donepezil or pharmaceutically acceptable salt in water/acidic medium;
(b) adding to the solution/suspension formed in step (a) an effective amount of pH dependent excipient; and
(c) formulating with excipient(s) into a solid oral dosage form.
Steps (a) and (b) may be optionally reversed

The pH dependent excipient may be selected from polacrilin potassium and eudragit E. The effective amount of pH dependent excipient may be defined by the weight ratio of donepezil to pH dependent excipient from 1:15 to 2:1, preferably in the range 1:1 to 1:6.

For the process of complexation of donepezil with polacrilin potassium optimization with reference to drug loading (ratio of active and resin), temperature and pH is carried out. The typical way of loading active ingredients onto an ion exchange resin is to dissolve an acidic or basic, ionizable active ingredients in water, and then mix it with a suitable ion exchange resin. In a similar fashion donepezil or pharmaceutically acceptable salt with pH dependent excipient may be complexed.

The taste of donepezil may be masked by admixing with Eudragit E in the presence of a solvent to form a drug-polymer interaction. Said interaction releases donepezil in the stomach at a pH below 5. The methods for preparing the drug-Eudragit E interaction ensures that physical effect, such as molecular inclusion, adsorption and granulation are present to significantly reduce the rate of release of the active ingredient and thereby effectively reduce the bitter taste of the active ingredient in the mouth.

The solvent used for the preparation of the compositions of the present invention may be a solvent wherein both the active ingredient and Eudragit E are soluble or suspended, namely ketones such as acetone, alcohols such as ethanol, dichloromethane, esters such as ethyl acetate and their mixtures with or without water. Alternatively, a solution of either the active ingredient or Eudragit E is prepared in a solvent or mixture of solvents and the solution is then combined with the remaining excipients. The solvent is not being removed, thus a simple wet or dry granulation process can be used for the commercial production.

More particularly, the present invention provides pharmaceutical composition of donepezil or pharmaceutically acceptable salt thereof, and an effective amount of polacrilin potassium as a taste masking agent, to suppress the release of donepezil in the pH environment of the oral cavity and increase the release of donepezil in acidic environment.

The process for preparing the pharmaceutical composition of the present invention comprises

(a) dissolving/dispersing donepezil or pharmaceutically acceptable salt in water/acidic medium;
(b) adding to the solution/suspension formed in (a) an effective amount of polacrillin potassium; and
(c) formulating with excipient(s) into a solid dosage form.
Steps (a) and (b) may be optionally reversed.

The effective amount of polacrilin potassium may be defined as weight ratio of donepezil to polacrilin potassium from 1:15 to 2:1, preferably in the range 1:1 to 1:6.

Typically the process of the present invention uses wet granulation process for the preparation of solid dosage forms containing Donepezil or salts thereof, which is one of the most economical methods. Said wet granulation process comprises:
dissolving the total quantity of donepezil or pharmaceutically acceptable salt thereof in water;
adding to the formed solution an effective amount of polacrilin potassium, as a taste masking agent, and wet granulating;
drying the wet granules;
sieving the dried granules to achieve the desired granule size;
forming a second blend by mixing the total quantity of any other optional excipient such as a binder, a disintegrant, a lubricant, a colorant and/or a glidant until uniform and
adding and mixing the dried granules with the second blend and
formulating the resulting mixture in a solid dosage form either by compressing it into a desired tablet form or by filling capsules or sachets.

The pharmaceutical compositions of the present invention have been tested in view of the taste masking efficiency. The composition of the present invention is better in taste and numbness. (Example 14)

The pharmaceutical compositions of the present invention are also characterized by excellent pharmacotechnical properties, such as homogeneity, flowability and compressibility. The solid dosage forms prepared by the above process exhibit excellent technical characteristics including disintegration time, dissolution rate, hardness, resistance to crushing, friability and stability, as better illustrated by the following measurements during the stage of the development of the products.

One of the most critical pharmacotechnical tests, is the Dissolution test as it is strongly correlated with the bioavailability of the product. The composition of the present invention suppresses release of donepezil in the pH of the oral cavity and increases release of donepezil in the acidic environment. (Example 12 and 15)

The validation process showed that the compositions and the manufacturing process are suitable in order to provide a repeatable and high quality product. No degradation products were observed during and after the procedure The pharmaceutical compositions of the present invention may also contain one or more additional formulation ingredients selected from a wide variety of excipients. According to the desired properties of the composition, any number of ingredients may be selected, alone or in combination, based upon their known uses in preparation of solid dosage form compositions. Such ingredients include, but are not limited to, fillers, binders, compression aids, disintegrants, glidants, lubricants, flavouring agents, water scavengers, colorants, sweetening agents, coating agents and preservatives.

The optional excipients must be compatible with donepezil or the salt thereof so that it does not interfere with it in the composition.

Diluents may be, for example, calcium carbonate, calcium phosphate dibasic, calcium phosphate tribasic, calcium sulfate, microcrystalline cellulose, microcrystalline silicified cellulose, powdered cellulose, dextrates, dextrose, fructose, lactitol, lactose anhydrous, lactose monohydrate, lactose dihydrate, lactose trihydrate, mannitol sorbitol, starch, pregelatinized starch, sucrose, talc, xylitol, maltose maltodextrin, maltitol. Binders may be, for example, acacia, alginic acid, carbomer, carboxymethylcellulose calcium, carboxymethylcellulose sodium, microcrystalline cellulose, powdered cellulose, ethyl cellulose, gelatin, liquid glucose, guar gum, hydroxyethyl cellulose, hydroxypropyl cellulose, hydroxypropylmethyl cellulose, maltodextrin, methylcellulose, polydextrose, polyethylene oxide, povidone, sodium alginate, starch paste, pregelatinized starch, sucrose, tragacanth, low-substituted hydroxypropyl cellulose, glucose, sorbitol. Suitable fillers are preferably selected from at least one of starch derivatives, such as corn starch, potato starch or rice starch; polysaccharides such as dextrins, maltodextrins, dextrates, microcrystalline cellulose, powdered cellulose, mixtures of microcrystalline cellulose and guar gum, coprocessed blends of microcrystalline cellulose; and polyhydric alcohols, such as xylitol and sorbitol. Disintegrants may be, for example, alginic acid, carbon dioxide, carboxymethylcellulose calcium, carboxymethylcellulose sodium, microcrystalline cellulose, powdered cellulose, croscarmelose sodium, crospovidone, sodium docusate, guar gum, hydroxypropyl cellulose, methylcellulose, polacrilin potassium, poloxamer, povidone, sodium alginate, sodium glycine carbonate, sodium laulyl sulfate, sodium starch glycolate, starch, pregelatinized starch, low-substituted hydroxypropyl cellulose. Glidants may be, for example, calcium silicate, powdered cellulose, starch, talc, colloidal silicon dioxide. Lubricants may be magnesium stearate, stearic acid, sodium stearyl fumarate, magnesium lauryl sulphate, talc, polyethylene glycol, and glyceryl behenate. Suitable sweeteners include sugars, such as sucrose, lactose and glucose; cyclamate and salts thereof; saccharin and salts thereof; and aspartame. Flavouring agents can be natural or synthetic flavours such as strawberry flavour, wild cherry flavour, green apple flavour, spearmint flavour, and peppermint flavour.

The following examples illustrate preferred embodiments in accordance with the present invention without limiting the scope or spirit of the invention

### EXAMPLES

### Example 1

### Donepezil Granules

Granules for 10 mg tablets

| Ingredients | g |
|---|---|
| Donepezil HCl | 0.50 |
| Polacrilin Potassium | 1.50 |
| Water | 200 ml |
| HCl | q.s. |

Granules were prepared according to the following manufacturing process: Donepezil HCL was dissolved into water under stirring. Polacrilin Potassium was admixed to said solution under stirring. The temperature of the solution was then adjusted to 40° C. The pH of the solution was adjusted to 4,5 using diluted HCl. Stirring of the solution continued for 4 to 8 hours. The solution was then filtered and the wetted mass was dried, passed though a sieve to achieve the desired granule size. The produced granules were mixed with suitable excipients.

### Example 2

### Donepezil Granules

Granules for 10 mg tablets

| Ingredients | g |
|---|---|
| Donepezil HCl | 1.00 |
| Eudragit E 100 | 3.40 |
| Talc | 1.00 |
| Mg Stearate | 0.20 |

Granules of Example 2 were prepared according to the following manufacturing process: Donepezil HCL was admixed with all the excipients and the formulation was blended to complete homogeneity. The above mixture was compacted, sized and granules were achieved. The granules were then compressed into tablets in a tableting machine with round punches.

### Example 3

### Donepezil Granules (Comparative Example)

| | |
|---|---|
| Calcium carbonate | 5.00 |
| Granules for 10 mg | |
| Talc | 0.50 g |
| tablets | |
| Mg Stearate | 0.10 |
| Ingredients | |
| Donepezil HCl | 0.50 |

### Example 4

### Donepezil Granules (Comparative Example)

Granules for 10 mg tablets

| Ingredients | g |
|---|---|
| Donepezil HCl | 0.50 |
| Dibasic Calcium Phosphate | 5.00 |
| Talc | 0.50 |
| Mg Stearate | 0.10 |

Granules of the formulation of example 3 and 4 were prepared using the procedure of Example 2.

### Example 5

### Donepezil Granules

Granules for 10 mg tablets

| Ingredients | g |
|---|---|
| Donepezil HCl | 0.50 |
| Polacrilin Potassium | 1.50 |
| Water | 20 ml |
| HCl | q.s. |

Granules of the formulation of example 5 were prepared using the procedure of Example 1.

### Example 6

### Donepezil Granules

Granules for 10 mg tablets

| Ingredients | g |
|---|---|
| Donepezil HCl | 1.00 |
| Polacrilin Potassium | 5.00 |
| HCL | 10 ml |

Granules of the above ingredients were prepared according to the following manufacturing process: Donepezil HCl was dissolved into HCL under stirring. Polacrilin Potassium was added into a mortar and granulated using the solution of Donepezil HCl. The solution was then filtered and the wetted mass was dried, passed though a sieve to achieve the desired granule size and the produced granules were mixed with suitable excipients.

### Example 7

### Donepezil Granules

Granules for 10 mg tablets

| Ingredients | g |
|---|---|
| Donepezil HCl | 1.00 |
| Polacrilin Potassium | 5.00 |
| MCC | 35.00 |
| HCl diluted | q.s. |
| Water | q.s. |

Granules of the above ingredients were prepared according to the following manufacturing process: Donepezil HCl was dissolved into water under stirring. Polacrilin Potassium was admixed to said solution under stirring. The temperature of the solution was then adjusted to 40° C. The pH of the solution was adjusted to 4,5 using diluted HCl. Stirring of the solution continued for about 4 to 6 hours. The solution was used for the granulation of MCC. The wetted mass was dried, passed though a sieve to achieve the desired granule size and the produced granules were mixed with suitable excipients.

### Example 8

### Donepezil Granules

Granules for 10 mg tablets

| Ingredients | g |
|---|---|
| Donepezil HCl | 3.15 |
| Eudragit E100 | 10.00 |
| MCC | 45.00 |
| Water | q.s. |
| Acetone | q.s. |

Granules of the above ingredients were prepared according to the following manufacturing process: MCC was granulated with water. Eudragit was dissolved into appropriate amount of Aceton under stirring. Donepezil HCl was added to the solution and admixed under stirring to form a homogenous suspension. The suspension was added to the MCC granules and granulated. The granules were dried, passed though a sieve to achieve the desired granule size. The produced granules were mixed with suitable excipients.

### Example 9

### Donepezil Granules

Granules for 10 mg tablets

| Ingredients | g |
|---|---|
| Donepezil HCl | 3.15 |
| Eudragit E | 10.00 |
| MCC | 45.00 |
| Water | q.s. |
| Acetone | q.s. |

Granules of the above ingredients were prepared according to the following manufacturing process: Eudragit was dissolved into Acetone and water and subsequently Donepezil was added under stirring to prepare a homogenous solution. The suspension was added to the MCC granules and re-granulated. The granules were dried, passed though a sieve to achieve the desired granule size. The produced granules were mixed with suitable excipients.

The same process was carried out using dichloromethane as a solvent.

### Example 10

### Orally Disintegrating Donepezil Tablets

| Ingredient | Mg per tablet |
|---|---|
| Donepezil/polacrilin | 190 |
| Mannitol | 268 |
| Sod. Citrate | 15 |
| Crosscarmelose sodium | 12 |
| Aspartame | 10 |
| Mg stearate | 5 |

The granules containing Donepezil HCl and Polacrilin produced according to any preceding example 1 and 5 to 7, were mixed with the remaining excipients and compressed to form tablets. The produced tablets were tested for hardness, friability, disintegration, and water content. All tests were performed according to European Pharmacopoeia 5.1 and were well within the specifications. Dissolution test in 900 ml water, 50 rpm Paddle Apparatus has been performed.

### Example 11

### Orally Disintegrating Donepezil Tablets

| Ingredient | Mg per tablet |
|---|---|
| Donepezil/Eudragit E | 193.8 |
| Mannitol | 254.2 |
| Sod. Citrate | 15 |
| Crosscarmelose sodium | 12 |
| Aspartame | 10 |
| Talc | 10 |
| Mg stearate | 5 |

The granules containing Donepezil HCl and Eudragit, produced according to any preceding example 2, 8 and 9, were mixed with the remaining excipients and compressed to form tablets. Tablets of high hardness were produced. The tablets were crushed and passed through a suitable sieve to produce granules of the desired size. The produced granules were mixed with suitable excipients.

### Example 12

### Comparison of Dissolution of Composition of the Present Invention with Aricept OD Under Different pH Conditions

### Dissolution Conditions:

### Apparatus: PhEur—Paddles

### Temperature: 37°±0.5° C.

### Rpm: 75 rpm

### Time: 30 min

Dissolution medium: 0.1N HCl, pH 4.5, pH 6.5

### Volume: 900 mL

(a) In 0.1 N HCl media:

| Product/Batch | Time (Min) | % Dissolved |
|---|---|---|
| Donepezil/Genepharm tabs | 5 | 83.5 |
| 10 mg/DDN002010 | 10 | 90.0 |
| | 15 | 92.6 |
| | 20 | 93.8 |
| | 30 | 95.4 |
| Aricept tabs 10 mg/6114504 | 5 | 83.9 |
| | 10 | 92.9 |
| | 15 | 96.5 |
| | 20 | 98.0 |
| | 30 | 98.9 |

(b) In pH 4.5 media:

| Product/Batch | Time (Min) | % Dissolved |
|---|---|---|
| Donepezil/Genepharm tabs | 5 | 36.5 |
| 10 mg/DDN002010 | 10 | 44.1 |
| | 15 | 49.1 |
| | 20 | 52.2 |
| | 30 | 56.5 |
| Aricept tabs 10 mg/6114504 | 5 | 85.0 |
| | 10 | 95.8 |
| | 15 | 99.5 |
| | 20 | 100.7 |
| | 30 | 102.2 |

(c) In pH 6.5 media:

| Product/Batch | Time (Min) | % Dissolved |
|---|---|---|
| Donepezil/Genepharm tabs | 5 | 58.8 |
| 10 mg/DDN002010 | 10 | 69.9 |
| | 15 | 75.4 |
| | 20 | 78.8 |
| | 30 | 83.5 |
| Aricept tabs 10 mg/6114504 | 5 | 87.9 |
| | 10 | 98.6 |
| | 15 | 101.9 |
| | 20 | 103.4 |
| | 30 | 103.9 |

### Example 13

### Chemical Stability of the Composition of the Present Invention

Donepezil tablets prepared according to the present invention were subjected to accelerated stability study at 40° C./75% RH. Comparison of the % purity of donepezil and related impurities analysed by HPLC is provided below:

| | | | | AFTER 3 | AFTER 6 |
|---|---|---|---|---|---|
| TESTS | METHODS | LIMITS | INITIAL | MONTHS | MONTHS |
| Assay | HPLC | 95.0-105.0% | 101.4% | 100.2% | 101.1% |
| Impurity A | HPLC | NMT 0.5% | ND | 0.01% | 0.02% |
| Impurity B | | NMT 0.5% | ND | <LOD | ND |
| Impurity C | | NMT 0.5% | 0.06% | 0.04% | 0.06% |
| Impurity D | | NMT 0.5% | 0.01% | ND | ND |

While the present invention has been described with respect to the particular embodiments, it will be apparent to those skilled in the art that various changes and modifications may be made in the invention without departing from the spirit and scope thereof, as defined in the appended claims.

### Example 14

The test was carried out by three examiners holding tablets for examination in their mouth, and then evaluated the degree of bitter taste and numbness in accordance of three grades. The granules were prepared as exemplified in Examples 1 to 4. The tablets were left to disintegrate for 15-30 seconds on the tongue to disintegrate and then were thrown out. Then the mouth was washed with water.

The marking “+++” used in the test represents the highest degree of bitterness/numbness. The results of this evaluation are shown in Table 1.

TABLE 1
Comparison of taste and numbness

| | | | Donepezil/ | | |
|---|---|---|---|---|---|
| | Donepezil/ | Donepezil/ | Calcium | Donepezil/ | Aricept |
| Taste | Eudragit E | Polacrilin | Carbonate | DCP | ODT |
| Bitterness | ++ | + | +++ | +++ | +++ |
| Numbness | ++ | + | +++ | +++ | +++ |

It is apparent from Table 1, that the composition according to the present invention using the complex between Donepezil and Polacrilin Potassium provides better taste masking in comparison with the other compositions and Aricept ODT.

### Example 15

For the dissolution method a Paddle Apparatus was used with 50 rpm, 37° C., time 30 min, while as a dissolution medium 900 ml of water was used.

TABLE 2
Comparison of % Release of donepezil

| | % RELEASE | | | |
|---|---|---|---|---|
| | | | DONEPEZIL/ | |
| TIME | ARICEPT | DONEPEZIL/ | CALCIUM | DONEPEZIL/ |
| (min) | ODT | POLACRILIN | CARBONATE | EUDRAGIT E |
| 5 | 66.8 | 79.3 | 93.1 | 84.4 |
| 10 | 86.7 | 89.7 | 99.0 | 93.2 |
| 15 | 93.2 | 94.6 | N/A | 98.4 |
| 20 | 96.9 | 97.0 | N/A | N/A |
| 30 | 104.7 | 99.4 | N/A | N/A |

As it can be concluded from the above table the release of the formulations under development was good and in all case follows the release of Aricept ODT but with low degree of bitterness and numbness.

1. A pharmaceutical composition for oral administration comprising donepezil or pharmaceutically acceptable salt thereof, and an effective amount of pH dependent excipient as a taste masking agent, to suppress the release of donepezil in the pH environment of the oral cavity and increase the release of donepezil in acidic environment.

2. A pharmaceutical composition according to claim 1, wherein effective amount of pH dependent excipient is weight ratio of donepezil or pharmaceutically acceptable salt thereof to pH dependent excipient ranging from 1:15 to 2:1.

3. A pharmaceutical composition according to claim 1, wherein said pH dependent excipient is an ion exchange resin.

4. A pharmaceutical composition according to claim 3, wherein said ion exchange resin is polacrilin potassium.

5. A pharmaceutical composition according to claim 4, wherein the weight ratio of donepezil or pharmaceutically acceptable salt thereof to polacrilin potassium is from 1:15 to 2:1.

6. A pharmaceutical composition according to claim 5, wherein the weight ratio of donepezil or pharmaceutically acceptable salt thereof to polacrilin potassium is 1:1 to 1 6.

7-9. (canceled)

10. A pharmaceutical composition according to claim 1, further comprising at least one optional excipient selected from diluents, binders, disintegrants, lubricants, glidants, taste modifiers, sweeteners, flow promoters and flavors.

11. A pharmaceutical composition according to claim 10, wherein said composition is in solid oral dosage form selected from tablet, oral dispersible tablet, soluble tablet, water dispersible tablet, sprinkles, chewable tablet, effervescent tablet, orally disintegrating tablet, powder for suspension, capsule or sachet.

12. A pharmaceutical composition according to claim 11 wherein said pharmaceutical composition is an orally disintegrating tablet.

13. A process for preparing solid oral dosage form of donepezil or pharmaceutically acceptable salt thereof with an effective amount of pH dependent excipient as a taste masking agent, to suppress the release of donepezil in the pH environment of the oral cavity and increase the release of donepezil in acidic environment, said process comprising
(a) dissolving/dispersing donepezil or pharmaceutically acceptable salt in water/acidic medium;
(b) adding to the solution/suspension formed in step (a) an effective amount of said pH dependent excipient; and
(c) formulating with excipient(s) into a solid oral dosage form.

14. A process according to claim 13, wherein effective amount of pH dependent excipient is weight ratio of donepezil or pharmaceutically acceptable salt thereof to pH dependent excipient ranging from 1:15 to 2:1.

15. A process according to claim 13, wherein said pH dependent excipient is an ion exchange resin.

16. A process according to claim 15, wherein said ion exchange resin is polacrilin potassium.

17. A process according to claim 16, wherein the weight ratio of donepezil or pharmaceutically acceptable salt thereof to polacrilin potassium is 1:1 to 1:6.

18-20. (canceled)

21. A process according to claim 13 wherein said dosage form is an orally disintegrating tablet.

22. Orally disintegrating tablet comprising Donepezil or pharmaceutically acceptable salt thereof and polacrilin potassium prepared according to claim 16.

23. (canceled)

24. A pharmaceutical composition for oral administration comprising donepezil or pharmaceutically acceptable salt thereof, and an effective amount of polacrilin potassium as a taste masking agent, wherein the weight ratio of donepezil or pharmaceutically acceptable salt thereof to polacrilin potassium is 1:1 to 1:6, to suppress the release of donepezil in the pH environment of the oral cavity and increase the release of donepezil in acidic environment.

25. A pharmaceutical composition according to claim 24, further comprising at least one optional excipient selected from diluents, binders, disintegrants, lubricants, glidants, taste modifiers, sweeteners, flow promoters and flavors.

26. A pharmaceutical composition according to claim 25, wherein said composition is in solid dosage form selected from tablet, oral dispersible tablet, soluble tablet, water dispersible tablet, sprinkles, chewable tablet, effervescent tablet, orally disintegrating tablet, powder for suspension, capsule or sachet.

27. A pharmaceutical composition according to claim 26, wherein said pharmaceutical composition is an orally disintegrating tablet.

28. A process for the preparation of a solid oral dosage form of donepezil or pharmaceutically acceptable salt thereof and an effective amount of polacrilin potassium as a taste masking agent, wherein the weight ratio of donepezil or pharmaceutically acceptable salt thereof to polacrilin potassium is 1:2 to 1:6, to suppress the release of donepezil in the pH environment of the oral cavity and increase the release of donepezil in acidic environment, said process comprising
(a) dissolving/dispersing donepezil or pharmaceutically acceptable salt in water/acidic medium;
(b) adding to the solution/suspension formed in (a) an effective amount of polacrillin potassium;
and
(c) formulating with excipient(s) into a solid dosage form.

29. A process according to claim 28 wherein said dosage form is an orally disintegrating tablet.

30. Orally disintegrating tablet comprising Donepezil or pharmaceutically acceptable salt thereof and polacrilin potassium prepared according to claim 28.
